(19) United States
(12) Patent Application Publication
(10) Pub. No.: US 2025/0249400 A1
(43) Pub. Date: Aug. 7, 2025

(54) Method and Application for Capturing and Converting Carbon Dioxide in Industrial Flue Gas

(71) Applicant: LINGHANGGUOCHUANG (BEIJING) TECHNOLOGY GROUP CO., LTD, Beijing (CN)

(72) Inventors: Enzhen DING, Ferntree Gully (AU); Angang LIU, Beijing (CN)

(21) Appl. No.: 18/264,087

(22) PCT Filed: May 17, 2023
(86) PCT No.: PCT/CN2023/094874

§ 371 Date: Aug. 3, 2023

(30) Foreign Application Priority Data
Aug. 27, 2022 (CN) 202211036073.7

(51) Int. Cl. B01D 53/62; B01D 53/26; B01D 53/76; C05C 3/00; C07C 68/04

(52) U.S. Cl. CPC B01D 53/62; B01D 53/265; B01D 53/76; C05C 3/00; C07C 68/04; B01D 2251/11; B01D 2257/502; B01D 2257/504; B01D 2258/0283

(57) ABSTRACT

The application discloses a method and application for capturing and converting carbon dioxide in industrial flue gas, pertaining to the field of carbon dioxide capture technology. The method comprises the following steps: introducing the industrial flue gas into the upper part of a cyclone separator to create a gas flow that rotates downward along the cyclone wall, simultaneously introducing fresh air into the lower part of the cyclone separator to create an air flow that rotates downward along the cyclone wall, and cooling the cyclone separator to make the industrial flue gas and air react to produce a mixed acid containing carbonic acid under conditions of temperature below 10° C. and pressure not less than 0.12 MPa, thereby capturing and converting carbon dioxide in the industrial flue gas. The entire process of this method is simple and efficient. It exhibits high safety, practical value.

### TECHNICAL FIELD

The present application relates to the technical field of carbon dioxide capture, more specifically, it involves a method and application for capturing and converting carbon dioxide in industrial flue gas.

### BACKGROUND ART

Currently, commonly used methods for capturing carbon dioxide in industrial settings include chemical absorption, physical adsorption, high-pressure liquefaction, molecular sieves, and other techniques. For instance, the patent application of publication number CN114602294A discloses a biphasic absorbent for CO₂ capture, which utilizes chemical absorption to capture carbon dioxide. Additionally, the patent application of authorization publication number CN214222307U discloses an integrated system for industrial waste gas carbon dioxide enrichment and liquefaction, employing high-pressure liquefaction for capturing and storing carbon dioxide.

However, in practical production processes, adsorption-based methods are complex and lack stability and safety. High-pressure liquefaction is prone to leaks and other safety accidents, resulting in high costs and difficulties in large-scale industrial applications.

### INVENTION DESCRIPTION

The purpose of the present application is to provide a method for capturing and converting carbon dioxide in industrial flue gas. The entire process of this method is simple and efficient, enabling effective capture of carbon dioxide in industrial flue gas at a low cost, while ensuring high safety and practical value.

Another purpose of present application is to provide an application of the method for capturing and converting carbon dioxide in industrial flue gas in the field of industrial flue gas purification.

The third purpose of present application is to provide an application of the method for capturing and converting carbon dioxide in the production of compound fertilizers from industrial flue gas conversion.

The technical issue of present application is addressed by implementing the following technical solutions.

First, the embodiments of the present application provide a method for capturing and converting carbon dioxide in industrial flue gas. The method comprises the following steps: introducing the industrial flue gas into the upper part of a cyclone separator to create a gas flow that rotates downward along the cyclone wall, simultaneously introducing fresh air into the lower part of the cyclone separator to create an air flow that rotates downward along the cyclone wall, and cooling the cyclone separator to make the industrial flue gas and air react to produce a mixed acid containing carbonic acid under conditions of temperature below 10° C. and pressure not less than 0.12 MPa, thereby capturing and converting carbon dioxide in the industrial flue gas.

Second, the embodiments of the present application provide an application of the method for capturing and converting carbon dioxide in industrial flue gas in the purification treatment of industrial flue gas.

Third, the embodiments of the present application provide an application of the method for capturing and converting carbon dioxide in the production of compound fertilizers from industrial flue gas conversion.

Compared with the existing technologies, the present application has at least the following advantages or beneficial effects:

As for the first aspect, the embodiments of the present application provide a method for capturing and converting carbon dioxide in industrial flue gas. This method involves introducing the industrial flue gas into the upper cylindrical part of a cyclone separator using a blower, nozzle, or other equipment and pressurizing it to inject it into the interior of the cyclone separator, creating a gas flow that rapidly rotates downward along the sidewall of the cyclone separator. Similarly, fresh air from the surroundings is introduced into the lower conical part of the cyclone separator using an air compressor, nozzle, or other equipment and pressurized to inject it into the interior of the cyclone separator, creating a gas flow that also rapidly rotates downward along the sidewall of the cyclone separator. During this process, the rotational speed of the air flow is greater than that of the flue gas flow. Under the effect of the pressure difference, the flue gas flow in the upper part is dragged and accelerated in a downward rotational motion. The cyclone separator is simultaneously cooled, ultimately forming a wall-seeking “super-heavy centrifugal cryogenic pressure field” inside the cyclone separator, with a temperature below 10° C. and a pressure not less than 0.12 MPa. Under these conditions, the moisture in the industrial flue gas condenses into droplets, and the chemical reaction pressure equilibrium parameters that are not easily achieved under normal conditions is altered by the pressure and temperature provided by “super-heavy centrifugal cryogenic pressure field”, making it possible for the carbon monoxide and carbon dioxide in the flue gas, along with the fresh air, to undergo a rapid reaction to produce carbonic acid. Similarly, the toxic substances such as nitrogen monoxide, nitrogen dioxide, and sulfur dioxide contained in the industrial flue gas can react to form corresponding acids. The relevant reactions are as follows:

$$2 C O + 2 C {O}_{2} + 4 {H}_{2} O + {O}_{2} \to 4 {H}_{2} C {O}_{3}$$

$$C {O}_{2} + {H}_{2} O \to {H}_{2} C {O}_{3}$$

$$4 N O + 2 {H}_{2} O + 3 {O}_{2} \to 4 H N {O}_{3}$$

$$4 N {O}_{2} + 2 {H}_{2} O + {O}_{2} \to 4 H N {O}_{3}$$

$$2 S {O}_{2} + 2 {H}_{2} O + {O}_{2} \to 2 {H}_{2} S {O}_{4}$$

The entire method process is simple, capable of effectively capturing carbon dioxide from industrial flue gas, while also purifying the industrial flue gas. It is cost-effective, environmentally friendly, and the process is safe, stable, and of high practical value.

As for the second aspect, the embodiments of the present application provide an application of the method for capturing and converting carbon dioxide in industrial flue gas in the purification treatment of industrial flue gas. The industrial flue gas can be subjected to multiple-stage treatments using this method to achieve different capture and purification effects.

As for the third aspect, the embodiments of the present application provide an application of the method for capturing and converting carbon dioxide in the production of compound fertilizers from industrial flue gas conversion. In the process of treating the industrial flue gas using this method, liquid nitrogen or ammonia water can be appropriately introduced into the cyclone separator, which, under the conditions provided by the “super-heavy centrifugal cryogenic pressure field,” allows it to react with the obtained carbonic acid to produce ammonium bicarbonate compound fertilizer containing a small amount of ammonium nitrate and ammonium sulfate. And later simple dehydration treatment of the compound fertilizer can yield agricultural compound fertilizer with an ammonium bicarbonate content of over 97%. The relevant reactions are as follows:

$${H}_{2} C {O}_{3} + N {H}_{3} \to N {H}_{4} H C {O}_{3}$$

$${H}_{2} S {O}_{4} + 2 N {H}_{3} \to {( N {H}_{4} )}_{2} S {O}_{4}$$

$$HN {O}_{3} + N {H}_{3} \to N {H}_{4} N {O}_{3}$$

### DESCRIPTION OF THE DRAWING

To provide a clearer understanding of the technical solution in the embodiment of the present application, a brief introduction to the drawing used in the embodiment will be provided. It should be understood that the drawing only represents certain embodiments of the present application and should not be considered as limiting the scope thereof. Ordinary skilled persons in the field can also obtain other related drawings based on the drawing without exercising inventive effort.

FIG. 1 represents a schematic diagram of a device that illustrates the method and for capturing and converting carbon dioxide in industrial flue gas provided in the present application.

The reference numbers are: 1. Shell; 11. Sealing baffle; 2. Cyclone separator; 21. Cylindrical section; 22. Conical section; 23. Cooling cavity; 3. The first cavity; 4. The second cavity; 5. The first air jet nozzle; 6. The second air jet nozzle; 7. Feeding pipe; 8. Collection pipe; 81. Exhaust pipe; 9. Collection box; 100. Circulating cooling device; 101. Inlet; 102. Outlet; 200. Medium-to-high-pressure blower; 300. Air compressor.

### DETAILED IMPLEMENTATION METHODS

In order to provide a clear description of the objectives, technical solutions, and advantages of the embodiment of the present application, the following will combine the drawing of the embodiment of the present application to describe the technical solutions in a clear and complete manner. It is evident that the described embodiment is only a part of the embodiments of the present application, not all of them. The components of the embodiment of the present application described and illustrated in the drawing can be arranged and designed in various configurations.

Conventional conditions or conditions recommended by manufacturers are used for embodiments where specific conditions are not indicated. Reagents or instruments not specified by the manufacturer are conventional products that can be obtained commercially.

Therefore, the detailed description of the embodiments of the present application provided in the drawing is not intended to limit the scope of the claimed invention, but only represents selected embodiments of the present application. Based on the embodiments disclosed in the present application, all other embodiments that ordinary skilled persons can obtain without exercising inventive effort are within the scope of protection of the present application.

It should be noted that similar reference numerals and letters in the following drawing represent similar items, so once an item is defined in one drawing, it does not need to be further defined or explained in subsequent drawing.

In the description of the embodiment of the present application, it should be noted that terms such as “up,” “down,” “inside,” “outside,” and the like indicating directions or positional relationships are based on the orientations or positional relationships shown in the drawings or the customary orientations or positional relationships when the inventive product is used. These terms are used for convenience of describing the present application and simplifying the description, and should not be construed as limiting the devices or components to specific orientations or constructing and operating in specific orientations, and thus should not be understood as limitations of the present application. Additionally, terms such as “first,” “second,” etc., are used for distinguishing purposes in the description and should not be construed as indicating or implying relative importance.

In the description of the embodiment of the present application, “multiple” represents at least two.

In the description of the embodiment of the present application, it is further clarified that unless otherwise explicitly specified or limited, terms such as “setting” and “connection” should be broadly interpreted. For example, it can refer to fixed connections, detachable connections or integral connections, mechanical connections or electrical connections, direct connections or indirect connections through intermediate media, or internal connections between two components. Ordinary skilled artisans in the field can understand the specific meanings of the aforementioned terms in the present application based on specific circumstances.

Unless there is a conflict, features in the embodiments of the present application can be combined with each other. Detailed descriptions of the present application will now be provided with reference to specific embodiments.

The embodiments of the present application provide a method for capturing and converting carbon dioxide in industrial flue gas. The method comprises the following steps: introducing the industrial flue gas into the upper part of a cyclone separator to create a gas flow that rotates downward along the cyclone wall, simultaneously introducing fresh air into the lower part of the cyclone separator to create an air flow that rotates downward along the cyclone wall, and cooling the cyclone separator to make the industrial flue gas and air react to produce a mixed acid containing carbonic acid under conditions of temperature below 10° C. and pressure not less than 0.12 MPa, thereby capturing and converting carbon dioxide in the industrial flue gas.

In the aforementioned embodiment, the industrial flue gas is introduced into the upper cylindrical part of a cyclone separator using a blower, nozzle, or other equipment and pressurized to inject it into the interior of the cyclone separator, creating a gas flow that rapidly rotates downward along the sidewall of the cyclone separator. Similarly, fresh air from the surroundings is introduced into the lower conical part of the cyclone separator using an air compressor, nozzle, or other equipment and pressurized to inject it into the interior of the cyclone separator, creating a gas flow that also rapidly rotates downward along the sidewall of the cyclone separator. During this process, the rotational speed of the air flow is greater than that of the flue gas flow. Under the effect of the pressure difference, the flue gas flow in the upper part is dragged and accelerated in a downward rotational motion. The cyclone separator is simultaneously cooled, ultimately forming a wall-seeking “super-heavy centrifugal cryogenic pressure field” inside the cyclone separator, with a temperature below 10° C. and a pressure not less than 0.12 MPa. Under these conditions, the moisture in the industrial flue gas condenses into droplets, and the chemical reaction pressure equilibrium parameters that are not easily achieved under normal conditions is altered by the pressure and temperature provided by “super-heavy centrifugal cryogenic pressure field”, making it possible for the carbon monoxide and carbon dioxide in the flue gas, along with the fresh air, to undergo a rapid reaction to produce carbonic acid. Similarly, the toxic substances such as nitrogen monoxide, nitrogen dioxide, and sulfur dioxide contained in the industrial flue gas can react to form corresponding acids.

The entire method process is simple, capable of effectively capturing carbon dioxide from industrial flue gas, while also purifying the industrial flue gas. It is cost-effective, environmentally friendly, and the process is safe, stable, and of high practical value.

Furthermore, in some embodiments of the present application, the industrial flue gas is introduced into the upper part of the cyclone separator using a medium-to-high-pressure blower, with a pressure ranging from 4,500 to 7,500 Pa; fresh air is introduced into the lower part of the cyclone separator using an air compressor, with a pressure ranging from 0.6 to 0.8 MPa.

Additionally, in some embodiments of the present application, the tangential velocity of the gas flow during rotation being 14 to 40 m/s, and the tangential velocity of the air flow during rotation being 60 to 150 m/s.

Moreover, in some embodiments of the present application, the cyclone separator is cooled using a cooling medium with a temperature ranging from 5° C. to −15° C.

In the aforementioned embodiments, by controlling the conditions such as pressure, airflow velocity, and cooling temperature, it is possible to create a wall-seeking “super-heavy centrifugal cryogenic pressure field” inside the cyclone separator, which further facilitates the reaction, enhances the effectiveness of carbon dioxide capture and conversion, and is more advantageous for industrial production applications.

Furthermore, in some embodiments of the present application, the above functions are achieved through the following devices: the shell 1 with the cyclone separator 2 arranged inside, and the bottom of the cyclone separator 2 is connected to the bottom of the shell 1; a cavity is formed between the cyclone separator 2 and the shell 1; the sealing baffle 11 is arranged in the cavity; the sealing baffle 11 is sleeved on the cyclone separator 2 and connected to the inner wall of the shell 1; the sealing baffle 11 divides the cavity into the first cavity 3 and the second cavity 4, wherein the first cavity 3 is located above the second cavity 4; the cooling cavity 23 is provided on the side wall of the cyclone separator 2, and a first injection module and a second injection module are also provided on the side wall of the cyclone separator 2, wherein the first injection module is located in the first cavity 3, and the second injection module is located in the second cavity 4.

In the aforementioned embodiments and during actual use, the industrial flue gas is introduced into first cavity 3 inside the shell 1 using a blower. The flue gas is then injected into the cyclone separator at a high speed through the first injection module, causing the flue gas to rotate downward along the inner wall of the cyclone separator 2. During this process, the cooling cavity 23 is filled with a cooling medium to cool the rotating flue gas. Simultaneously, fresh air is introduced into second cavity 4 and injected into the lower part of the cyclone separator 2 through the second injection module, causing the fresh air to rotate along the inner wall of the cyclone separator as well. At this point, the upper part of the conical section consists of a high-speed downward rotating stream of flue gas, while the lower part consists of a high-speed downward rotating stream of air. The pressure difference generated by the high-speed air flow in the lower part drags the upper part of the flue gas, causing it to rotate more rapidly along the inner wall of the cyclone separator 2 in a downward motion. Finally, under the action of the cooling medium in the cooling cavity 23, a wall-seeking “super-heavy centrifugal cryogenic pressure field” is formed inside the cyclone separator 2. Under these conditions, the moisture in the industrial flue gas condenses into droplets, and the chemical reaction pressure equilibrium parameters that are not easily achieved under normal conditions is altered by the pressure and temperature provided by “super-heavy centrifugal cryogenic pressure field”, making it possible for the carbon monoxide and carbon dioxide in the flue gas, along with the fresh air, to undergo a rapid reaction to generate carbonic acid. Similarly, the toxic substances such as nitrogen monoxide, nitrogen dioxide, and sulfur dioxide contained in the industrial flue gas can react to form corresponding acids. If liquid nitrogen or ammonia water is appropriately introduced into the cyclone separator 2, which, under the conditions provided by the “super-heavy centrifugal cryogenic pressure field,” allows it to react with the obtained carbonic acid to produce ammonium bicarbonate compound fertilizer containing a small amount of ammonium nitrate and ammonium sulfate. And later simple dehydration treatment of the compound fertilizer can yield agricultural compound fertilizer with an ammonium bicarbonate content of over 97%.

The entire equipment has a simple structure, capable of capturing carbon dioxide from flue gas while purifying the gas. Additionally, it can further convert the captured carbon dioxide into composite nitrogen fertilizer, achieving resource recovery and reuse. During operation, the entire process is safe, stable, simple, and efficient, with low costs and high stability. It exhibits characteristics such as energy conservation, emission reduction, economic and environmental sustainability, and resource conservation, demonstrating high practical value.

Furthermore, in some embodiments of the present application, the first injection module comprises multiple first air jet nozzles 5, and the second injection module comprises multiple second air jet nozzles 6, wherein the first air jet nozzle 5 and the second air jet nozzle 6 are both arranged through the side wall of the cyclone separator 2; the inlet ends of the first air jet nozzle 5 and the second air jet nozzle 6 are located inside the cavity, and the outlet ends are located inside the cyclone separator 2 and positioned lower than the inlet ends.

In the aforementioned embodiments, the inclination of multiple first air jet nozzles 5 and multiple second air jet nozzles 6 towards the interior of the cyclone separator 2 facilitates a better realization of high-speed downward rotation of the airflow along the inner wall of the cyclone separator 2. Specifically, the airflow velocity from the second nozzles is greater than that from the first airflow nozzle 5, enabling the acceleration and drag of the upper smoke flow through the pressure difference.

Additionally, in some embodiments of the present application, the cyclone separator 2 comprises the cylindrical section 21 and the conical section 22, wherein the larger end of the conical section 22 is connected to the end of the cylindrical section 21, while the smaller end of the conical section 22 is connected to the bottom of the shell 1 and in communication with the outside; the sealing baffle 11 is positioned between the cylindrical section 21 and the conical section 22, with the cylindrical section 21 located inside the first cavity 3 and the conical section located inside the second cavity 4.

In the aforementioned embodiments, such configuration enables a more rational device structure and contributes to enhancing the capture efficiency.

Moreover, in some embodiments of the present application, the aforementioned devices also comprise the circulating cooling device 100, which is provided with the outlet 102 and the Inlet 101; the outlet 102 is connected to the bottom of the cooling cavity 23 via a pipeline, while the Inlet 101 is connected to the top of the cooling cavity 23 via a pipeline.

In the aforementioned embodiments, by introducing fresh air into second cavity 4 through the feeding pipe 7, and simultaneously adding liquid nitrogen or ammonia water, the captured carbon dioxide can be reacted and converted into ammonium bicarbonate compound fertilizer, achieving the recycling of resources.

The embodiments of the present application also provide an application of the method for capturing and converting carbon dioxide in industrial flue gas in the purification treatment of industrial flue gas.

The embodiments of the present application also provide an application of the method for capturing and converting carbon dioxide in the production of compound fertilizers from industrial flue gas conversion

Further detailed description of the features and performance of the present application is provided below, in conjunction with the embodiments:

### Embodiment 1

This embodiment provides a method for capturing and converting carbon dioxide in industrial flue gas. The method comprises the following steps: introducing industrial flue gas into the upper part of the cyclone separator using a medium-to-high-pressure blower, maintaining a pressure of 4,500 Pa to make the gas flow form a swirling airflow along the cylindrical wall of the cyclone separator rotating downwards at a tangential velocity of 14 m/s; and introducing fresh air into the lower part of the cyclone separator using an air compressor under a pressure of 0.6 MP to make the air form a swirling airflow along the cylindrical wall of the cyclone separator rotating downwards at a tangential velocity of 60 m/s. The cyclone separator is cooled using a cooling medium with a temperature of 5° C. to make the industrial flue gas and air undergo a reaction to generate a mixed acid containing carbonic acid under the conditions of a temperature below 10° C. and a pressure not less than 0.12 MPa, offering an approach for the capture and conversion of carbon dioxide in the industrial flue gas.

### Embodiment 2

This embodiment provides a method for capturing and converting carbon dioxide in industrial flue gas. The method comprises the following steps: introducing industrial flue gas into the upper part of the cyclone separator using a medium-to-high-pressure blower, maintaining a pressure of 7,500 Pa to make the gas flow form a swirling airflow along the cylindrical wall of the cyclone separator rotating downwards at a tangential velocity of 40 m/s; and introducing fresh air into the lower part of the cyclone separator using an air compressor under a pressure of 0.8 MP to make the air form a swirling airflow along the cylindrical wall of the cyclone separator rotating downwards at a tangential velocity of 150 m/s. The cyclone separator is cooled using a cooling medium with a temperature of −15° C. to make the industrial flue gas and air undergo a reaction to generate a mixed acid containing carbonic acid under the conditions of a temperature below 10° C. and a pressure not less than 0.12 MPa, offering an approach for the capture and conversion of carbon dioxide in the industrial flue gas.

### Embodiment 3

This embodiment provides a method for capturing and converting carbon dioxide in industrial flue gas. The method comprises the following steps: introducing industrial flue gas into the upper part of the cyclone separator using a medium-to-high-pressure blower, maintaining a pressure of 6,000 Pa to make the gas flow form a swirling airflow along the cylindrical wall of the cyclone separator rotating downwards at a tangential velocity of 30 m/s; and introducing fresh air into the lower part of the cyclone separator using an air compressor under a pressure of 0.8 MP to make the air form a swirling airflow along the cylindrical wall of the cyclone separator rotating downwards at a tangential velocity of 120 m/s. The cyclone separator is cooled using a cooling medium with a temperature of −10° C. to make the industrial flue gas and air undergo a reaction to generate a mixed acid containing carbonic acid under the conditions of a temperature below 10° C. and a pressure not less than 0.12 MPa, offering an approach for the capture and conversion of carbon dioxide in the industrial flue gas.

### Embodiment 4

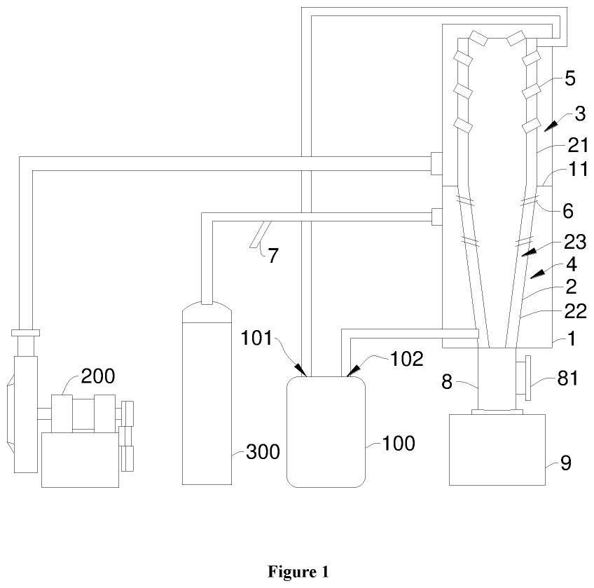

Please refer to FIG. 1. This embodiment provides a method for capturing and converting carbon dioxide in industrial flue gas, which is implemented through the following devices:

The shell 1 with the cyclone separator 2 arranged inside, and the bottom of the cyclone separator 2 is connected to the bottom of the shell 1; a cavity is formed between the cyclone separator 2 and the shell 1; the sealing baffle 11 is arranged in the cavity; the sealing baffle 11 is sleeved on the cyclone separator 2 and connected to the inner wall of the shell 1; the sealing baffle 11 divides the cavity into the first cavity 3 and the second cavity 4, wherein the first cavity 3 is located above the second cavity 4; the cooling cavity 23 is provided on the side wall of the cyclone separator 2, and a first injection module and a second injection module are also provided on the side wall of the cyclone separator 2, wherein the first injection module is located in the first cavity 3, and the second injection module is located in the second cavity 4.

The first injection module comprises multiple first air jet nozzles 5, and the second injection module comprises multiple second air jet nozzles 6, wherein the first air jet nozzle 5 and the second air jet nozzle 6 are both arranged through the side wall of the cyclone separator 2; the inlet ends of the first air jet nozzle 5 and the second air jet nozzle 6 are located inside the cavity, and the outlet ends are located inside the cyclone separator 2 and positioned lower than the inlet ends.

The cyclone separator 2 comprises the cylindrical section 21 and the conical section 22, wherein the larger end of the conical section 22 is connected to the end of the cylindrical section 21, while the smaller end of the conical section 22 is connected to the bottom of the shell 1 and in communication with the outside; the sealing baffle 11 is positioned between the cylindrical section 21 and the conical section 22, with the cylindrical section 21 located inside the first cavity 3 and the conical section located inside the second cavity 4.

The circulating cooling device 100 is also included, which is provided with the outlet 102 and the Inlet 101; the outlet 102 is connected to the bottom of the cooling cavity 23 via a pipeline, while the Inlet 101 is connected to the top of the cooling cavity 23 via a pipeline.

The specific method is as follows: introducing the industrial flue gas into the first cavity 3 using the medium-to-high-pressure blower 200 at a pressure of 6,000 Pa; injecting the flue gas into the cyclone separator 2 through the first air jet nozzles 5 to form a gas flow that rotates from the upper part and downward along the cylindrical wall of the cyclone separator 2 at a tangential velocity of 30 m/s; simultaneously, introducing fresh air into second cavity 4 using the air compressor 300 at a pressure of 0.8 MPa; injecting the fresh air into the cyclone separator 2 through the second air jet nozzles 6 to form an airflow that rotates from the lower part and downward along the cylindrical wall of the cyclone separator 2 at a tangential velocity of 120 m/s; cooling the cyclone separator 2 using the circulating cooling device 100 by introducing a cooling medium with a temperature of −10° C. into the cooling cavity 23 via the outlet 102 and recycling the cooling medium through the Inlet 101 of the cooling cavity 23 to finally make the industrial flue gas and air react under conditions of temperature below 10° C. and pressure not less than 0.12 MPa to generate a mixed acid including carbonic acid, offering an approach for the capture and conversion of carbon dioxide in the industrial flue gas.

### Experimental Case

Tire combustion smoke is treated using the method for capturing and converting carbon dioxide in industrial flue as provided in embodiment 4 of the present application. Detailed changes in the state data of the smoke before and after the treatment is detected and recorded. The specific projects and results are shown in table 1.

TABLE 1

| | | | Before | After | Reduction |
|---|---|---|---|---|---|
| Project/Name | | | Treatment | Treatment | Rate/% |
| Exhaust Emission (wet basis) | | NM3/h | 521 | 242 | 53.6 |
| Exhaust Emission (dry basis) | | | 451 | 235 | 47.9 |
| Exhaust Moisture Content | | Vol/% | 13.5 | 3 | 89.7 |
| Main Components | CO₂ | Vol/% | 3.6 | 2.6 | 66.5 |
| Analysis of Exhaust: | O₂ | Vol/% | 16.8 | 17.7 | — |
| | CO | Vol/% | 0 | 0 | — |
| | N₂ | Vol/% | 79.6 | 79.7 | — |

| SOx Concentration | vol | 562 | 15 | 98.8 |
|---|---|---|---|---|
| | ppm | | | |
| SOx Emission | NM3/h | — | 0.003 | — |
| NOx Concentration | vol | 37 | 15 | 81.2 |
| NOx Emission Concentration | ppm | Converted | 41 | — |
| | | Value at O₂ | | |
| | | 12% | | |
| HCl Concentration | mg/NM3 | 134 | 34 | 88.2 |
| HCl Emission Concentration | | Converted | 92 | — |
| | | Value at O2 | | |
| | | 12% | | |
| Dust Concentration | g/NM³ | 16.2 | 0.02 | 99.9 |
| Dust Emission Rate | | Converted | 0.05 | — |

Value at O₂
12%

The results demonstrate that by applying the method for capturing and converting carbon dioxide in industrial flue gas provided in the present application, the total exhaust gas emissions from tire combustion are reduced by 53.600. Vapor is condensed into liquid water with a recovery rate of 89.70%. CO₂ is converted to H₂CO₃ at a rate as high as 66.50%. SO_{X} is converted to H₂SO₄ with a conversion rate of 99.80%. NOx is converted to HINO₃ with a conversion rate of 81.200. The effects are evident and significant.

Based on aforementioned, the embodiments of the present application provide a method and application for capturing and converting carbon dioxide in industrial flue gas. This method involves introducing the industrial flue gas into the upper cylindrical part of a cyclone separator using a blower, nozzle, or other equipment and pressurizing it to inject it into the interior of the cyclone separator, creating a gas flow that rapidly rotates downward along the sidewall of the cyclone separator. Similarly, fresh air from the surroundings is introduced into the lower conical part of the cyclone separator using an air compressor, nozzle, or other equipment and pressurized to inject it into the interior of the cyclone separator, creating a gas flow that also rapidly rotates downward along the sidewall of the cyclone separator. During this process, the rotational speed of the air flow is greater than that of the flue gas flow. Under the effect of the pressure difference, the flue gas flow in the upper part is dragged and accelerated in a downward rotational motion. The cyclone separator is simultaneously cooled, ultimately forming a wall-seeking “super-heavy centrifugal cryogenic pressure field” inside the cyclone separator, with a temperature below 10° C. and a pressure not less than 0.12 MPa. Under these conditions, the moisture in the industrial flue gas condenses into droplets, and the chemical reaction pressure equilibrium parameters that are not easily achieved under normal conditions is altered by the pressure and temperature provided by “super-heavy centrifugal cryogenic pressure field”, making it possible for the carbon monoxide and carbon dioxide in the flue gas, along with the fresh air, to undergo a rapid reaction to produce carbonic acid. Similarly, the toxic substances such as nitrogen monoxide, nitrogen dioxide, and sulfur dioxide contained in the industrial flue gas can react to form corresponding acids. The entire method process is simple, capable of effectively capturing carbon dioxide from industrial flue gas, while also purifying the industrial flue gas. It is cost-effective, environmentally friendly, and the process is safe, stable, and of high practical value. It is also can be applied to flue gas purification and compound fertilizer production from industrial flue gas conversion.

The aforementioned are merely preferred embodiments of the present application and should not be construed as limiting the scope of the present application. For those skilled persons in this field, the present application may subject to various modifications and changes. Any modifications, equivalent substitutions, improvements, etc., made within the spirit and principles of the present application should be included within the scope of protection of the present application.

1. A method for capturing and converting carbon dioxide in industrial flue gas, characterized by comprising the following steps: introducing the industrial flue gas into the upper part of a cyclone separator to create a gas flow that rotates downward along the cyclone wall, simultaneously introducing fresh air into the lower part of the cyclone separator to create an air flow that rotates downward along the cyclone wall, and cooling the cyclone separator to make the industrial flue gas and air react to generate a mixed acid containing carbonic acid under conditions of temperature below 10° C. and pressure not less than 0.12 MPa, offering an approach for the capture and conversion of carbon dioxide in the industrial flue gas.

2. The method for capturing and converting carbon dioxide in industrial flue as claimed in claim 1 is characterized by introducing the industrial flue gas into the upper part of the cyclone separator using a medium-to-high-pressure blower, with a pressure ranging from 4,500 to 7,500 Pa; introducing fresh air into the lower part of the cyclone separator using an air compressor, with a pressure ranging from 0.6 to 0.8 MPa.

3. The method for capturing and converting carbon dioxide in industrial flue gas as claimed in claim 1 is characterized by the tangential velocity of the gas flow during rotation being 14 to 40 m/s, and the tangential velocity of the air flow during rotation being 60 to 150 m/s.

4. The method for capturing and converting carbon dioxide in industrial flue gas as claimed in claim 1 is characterized by cooling the cyclone separator using a cooling medium with a temperature ranging from 5° C. to −15° C.

5. The method for capturing and converting carbon dioxide in industrial flue gas as claimed in claim 1 is characterized by being implemented through the following devices: a shell with a cyclone separator arranged inside, and the bottom of the cyclone separator is connected to the bottom of the shell; a cavity is formed between the cyclone separator and the shell; a sealing baffle is arranged in the cavity; the sealing baffle is sleeved on the cyclone separator and connected to the inner wall of the shell; the sealing baffle divides the cavity into a first cavity and a second cavity, wherein the first cavity is located above the second cavity; a cooling cavity is provided on the side wall of the cyclone separator, and a first injection module and a second injection module are also provided on the side wall of the cyclone separator, wherein the first injection module is located in the first cavity, and the second injection module is located in the second cavity.

6. A method for capturing and converting industrial flue gas carbon dioxide as claimed in claim 5 is characterized in that the first injection module comprises multiple first air jet nozzles, and the second injection module comprises multiple second air jet nozzles, wherein the first air jet nozzles and the second air jet nozzles are both arranged through the side wall of the cyclone separator; the inlet ends of the first air jet nozzles and the second air jet nozzles are located inside the cavity, and the outlet ends are located inside the cyclone separator and positioned lower than the inlet ends.

7. A method for capturing and converting industrial flue gas carbon dioxide as claimed in claim 5 is characterized in that the cyclone separator comprises a cylindrical section and a conical section, wherein the larger end of the conical section is connected to the end of the cylindrical section, while the smaller end of the conical section is connected to the bottom of the shell and in communication with the outside; the sealing baffle is positioned between the cylindrical section and the conical section, with the cylindrical section located inside the first cavity and the conical section located inside the second cavity.

8. A method for capturing and converting industrial flue gas carbon dioxide as claimed in claim 5 is characterized in that the apparatus further comprises a circulating cooling device, which is provided with an outlet and an inlet; the outlet is connected to the bottom of the cooling cavity via a pipeline, while the inlet is connected to the top of the cooling cavity via a pipeline.

9. The application of the method for capturing and converting carbon dioxide in industrial flue gas as claimed in claim 8 in industrial flue gas purification treatment.

10. The application of the method for capturing and converting carbon dioxide in industrial flue gas as claimed in claim 8 in the production of compound fertilizers from industrial flue gas conversion.
